(19) United States
(12) Patent Application Publication
(10) Pub. No.: US 2012/0004614 A1
(43) Pub. Date: Jan. 5, 2012

(54) Production Process for a Microneedle Arrangement and Corresponding Microneedle Arrangement and Use

(75) Inventors: Michael Stumber, Korntal-Muenchingen (DE); Frank Schatz, Kornwestheim (DE)

(73) Assignee: Robert Bosch GmbH, Stuttgart (DE)

(21) Appl. No.: 13/171,942

(22) Filed: Jun. 29, 2011

(30) Foreign Application Priority Data
Jul. 2, 2010 (DE) 10 2010 030 864.1

(51) Int. Cl. A61M 5/00; C23F 1/04

(52) U.S. Cl. 604/173; 216/11

(57) ABSTRACT

A production process for a microneedle arrangement and a corresponding microneedle arrangement as well as a use for it is disclosed. The process has the following steps: forming an etching mask in grid form, with grid bars with corresponding grid crossing regions and grid openings in between on a substrate; carrying out an etching process to form the microneedle arrangement on the substrate using the etching mask and removing the etching mask. The etching mask in grid form has at least some of the grid crossing regions flat reinforcing regions, which extend beyond the grid bars.

This application claims priority under 35 U.S.C. §119 to German patent application no. DE 10 2010 030 864.1, filed Jul. 2, 2010 in Germany, the disclosure of which is incorporated herein by reference in its entirety.

### BACKGROUND

The present disclosure relates to a production process for a microneedle arrangement and to a corresponding microneedle arrangement as well as to a use for it.

Although it can be applied to any micromechanical components, the present disclosure and the background on which it is based are explained with regard to micromechanical components in silicon technology.

Microneedle arrangements, which for example comprise microneedles of porous silicon, are used in the area of “transdermal drug delivery” as a supplement to medicament plasters, as a carrier of a vaccine and also for obtaining body fluid (known as “transdermal fluid”) for the diagnosis and analysis of body parameters (for example glucose, lactate, . . . ).

Medicament plasters (transdermal patches) for small molecules (for example nicotine) are widely known. To extend the application area for such transdermal applications of active substances, use is made of so-called chemical enhancers or various physical methods (ultrasound, heat pulses), which help to overcome the protective covering that is the skin.

A further method for this is mechanical perforation of the outer layers of skin (stratum corneum) by fine porous microneedles, combined with the administration of an active substance, preferably via an active substance plaster in which the microneedles may already be integrated, or via a dosing device, which makes a specific release (bolus, pause, increase, . . . ) of active substances possible.

DE 10 2006 028 781 A1 discloses a process for producing porous microneedles arranged in an array on a silicon substrate for the transdermal administration of medicaments. The process comprises forming on the front side of a semiconductor substrate a microneedle arrangement with a plurality of microneedles, which rise up from a supporting region of the semiconductor substrate, as well as partially porosifying the semiconductor substrate to form porous microneedles, the porosifying being performed from the front side of the semiconductor substrate and a porous reservoir being formed.

DE 10 2006 028 914 A1 discloses a process for producing microneedles from porous material, a coating of silicon being applied over a microneedle arrangement while the tips of the needles remain uncovered, after which a process of porosifying the microneedles is carried out.

DE 10 2006 040 642 A1 discloses a microneedle arrangement for placement in the skin for the purpose of transdermal application of pharmaceuticals.

FIGS. 8a,b are schematic representations for the explanation of a production process given by way of example for a microneedle arrangement, to be precise FIG. 8a is a plan view of an etching grid and FIG. 8b is a cross-sectional view of the etching grid and of the microneedle arrangement resulting from it along the line A-A′ from FIG. 8a.

In FIG. 8, reference sign 10 denotes an etching mask, which is applied to a silicon substrate 1. The etching mask 10 is, for example, an oxide mask, which is produced by a suitable photolithographic process on the silicon substrate 1 after a full-area oxidation or oxide deposition.

The etching mask 10 has the form of a regular square etching grid with horizontal grid bars 100 and vertical grid bars 110 orthogonal thereto. Reference sign 10a denotes a respective grid crossing region between the grid bars 100 and 110. Reference sign 10b denotes a respective grid opening, through which an etching medium can pass to the silicon substrate 1 during the etching process, in order to porosify it and thus form the microneedles.

The structuring of a microneedle arrangement 20 with a plurality of microneedles 200 arranged in the form of a matrix corresponding to the etching grid 10 takes place by an anisotropic etching process known per se (for example DRIE) and an isotropic plasma etching process. The anisotropic etching process and the isotropic etching process may either be carried out once, one after the other, that is to say first anisotropic and then isotropic, or else in an alternating manner, for example anisotropic-isotropic-anisotropic-isotropic- . . . and so on.

After the etching, the microneedles 200 remain under the grid crossing regions 10a. In the case of the etching mask 10 that is used according to FIGS. 8a,b, a supporting region la of the semiconductor substrate 1 is also left behind at the foot of the microneedles 200.

After the etching, the etching mask 10 spans the microneedle arrangement 20 and is suspended over the substrate 1 in a peripheral region not represented. The exposure of the microneedle arrangement 20 by removing the etching mask takes place by an oxide etching step. Porosifying can then be performed, if desired, in a further known etching step.

A functional aspect of a microneedle arrangement is that the needles are intended to pierce the skin as well as possible, i.e. they should be as pointed as possible, but also must not be too close, since otherwise an undesired “Fakir effect” occurs, that is to say hindered penetration of the needles into the skin. On the other hand, a desired effect, for example a great transfer of active substance, often requires as many needles as possible and correspondingly many piercings of the skin. If, however, this is at the expense of a large area, the costs increase rapidly, since they are in linear proportion to the wafer area that is required for a selected process.

### SUMMARY

The production process according to the disclosure for a microneedle arrangement and the corresponding microneedle arrangement as well as the use have the advantage that the grid crossing regions of the etching mask are reinforced in terms of their surface area in comparison with the grid bars, in order in this way to produce thicker and more stable microneedles in the etching process.

If, for example, microneedles of different heights are placed next to one another within a microneedle arrangement, the longer microneedles can penetrate the skin first, and the somewhat shorter ones then follow into the already penetrated skin, which makes the piercing process more reliable, more effective and more stable. Patterns which can for example be used for tattooing can also be generated.

One effect of using the etching masks according to the disclosure is that inhomogeneities on the substrate surface after the etching processes can be corrected, so that a uniform microneedle array is obtained over the wafer, which is accompanied by an increased yield.

The features set forth in the disclosure make a specifically adaptable height pattern of the microneedles possible within a microneedle arrangement, which can be adapted according to the application, and whereby not only the piercing characteristics but also the stability of the needles can be adapted to requirements.

The features presented in the dependent claims relate to advantageous developments and improvements of the relevant subject matter of the disclosure.

### BRIEF DESCRIPTION OF THE DRAWINGS

Exemplary embodiments of the disclosure are explained in more detail in the description which follows and are represented in the drawing, in which:

FIGS. 1a,b show schematic representations for the explanation of a first embodiment of the production process according to the disclosure for a microneedle arrangement, to be precise FIG. 1a shows a plan view of an etching grid and FIG. 1b shows a cross-sectional view of the etching grid and of the microneedle arrangement resulting from it along the line A-A′ from FIG. 1a;

FIGS. 2a,b show schematic representations for the explanation of a second embodiment of the production process according to the disclosure for a microneedle arrangement, to be precise FIG. 2a shows a plan view of an etching grid and FIG. 2b shows a cross-sectional view of the etching grid and of the microneedle arrangement resulting from it along the line A-A′ from FIG. 2a;

FIGS. 3a,b show schematic representations for the explanation of a third embodiment of the production process according to the disclosure for a microneedle arrangement, to be precise FIG. 3a shows a plan view of an etching grid and FIG. 3b shows a cross-sectional view of the etching grid and of the microneedle arrangement resulting from it along the line A-A′ from FIG. 3a;

FIGS. 4a,b show schematic representations for the explanation of a fourth embodiment of the production process according to the disclosure for a microneedle arrangement, to be precise FIG. 4a shows a plan view of an etching grid and FIG. 4b shows a cross-sectional view of the etching grid and of the microneedle arrangement resulting from it along the line A-A′ from FIG. 4a;

FIGS. 5a,b show schematic representations for the explanation of a fifth embodiment of the production process according to the disclosure for a microneedle arrangement, to be precise FIG. 5a shows a plan view of an etching grid and FIG. 5b shows a cross-sectional view of the etching grid and of the microneedle arrangement resulting from it along the line A-A′ from FIG. 5a;

FIG. 6 shows a plan view of an etching grid for the explanation of a sixth embodiment of the production process according to the disclosure for a microneedle arrangement;

FIG. 7 shows a plan view of an etching grid for the explanation of a seventh embodiment of the production process according to the disclosure for a microneedle arrangement; and

FIGS. 8a,b show schematic representations for the explanation of a production process for a microneedle arrangement given by way of example, to be precise FIG. 8a shows a plan view of an etching grid and FIG. 8b shows a cross-sectional view of the etching grid and of the microneedle arrangement resulting from it along the line A-A′ from FIG. 8a.

### DETAILED DESCRIPTION

In the figures, the same reference signs denote elements that are the same or functionally the same.

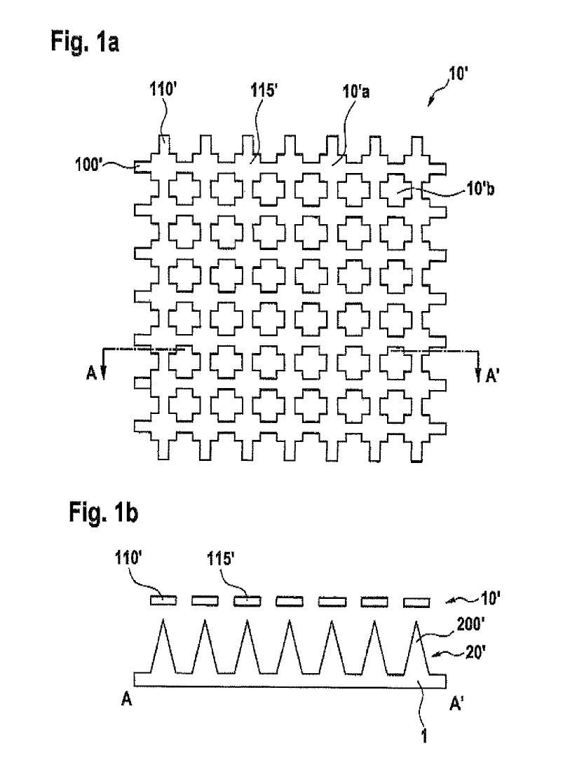

FIGS. 1a,b are schematic representations for the explanation of a first embodiment of the production process according to the disclosure for a microneedle arrangement, to be precise FIG. 1a is a plan view of an etching grid and FIG. 1b is a cross-sectional view of the etching grid and of the microneedle arrangement resulting from it along the line A-A′ from FIG. 1a.

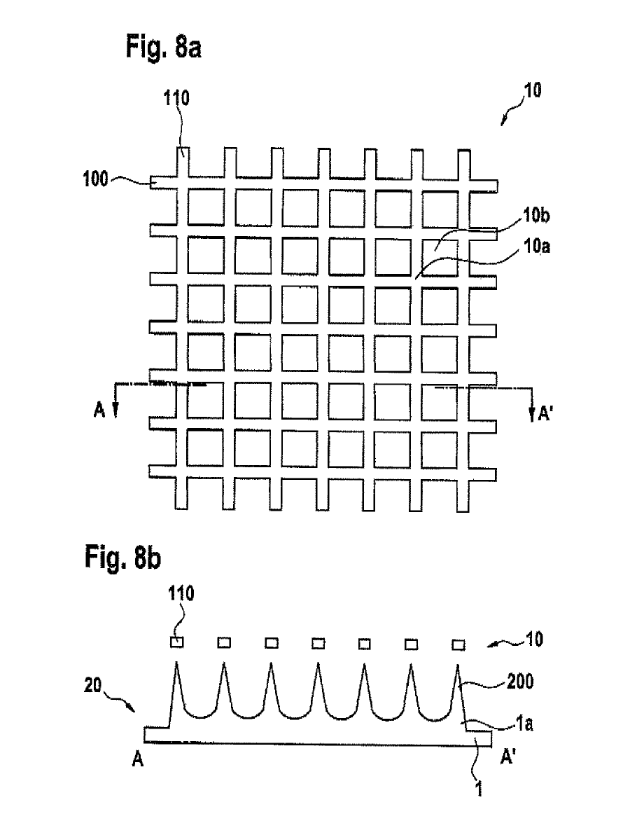

In the case of the first embodiment, reference sign 10′ denotes an etching mask, which like the etching mask 10 according to FIGS. 8a,b comprises a regular orthogonal grid of horizontal grid bars 100′ and vertical grid bars 110′. The grid crossing regions are denoted by reference sign 10′a and the grid openings are denoted by reference sign 10′b.

By contrast with the etching mask 10 described above, the etching mask 10′ has at the grid crossing regions 10′a square reinforcing regions 115′, which have a greater cross section than the grid bars 100′, 110′ and which extend beyond the grid bars 100′, 110′ into the grid openings 10′b.

If the anisotropic/isotropic etching process already described in connection with FIG. 8 is applied to a silicon substrate 1 which is covered by the etching mask 10′ of oxide, the form of microneedles represented in FIG. 1b is obtained, comprising thicker, more stable microneedles 200′ than the microneedles 200 in FIG. 8b. In particular, the supporting region 1a according to FIG. 8b has almost completely disappeared in the case of the microneedle arrangement 20′ according to FIG. 1b.

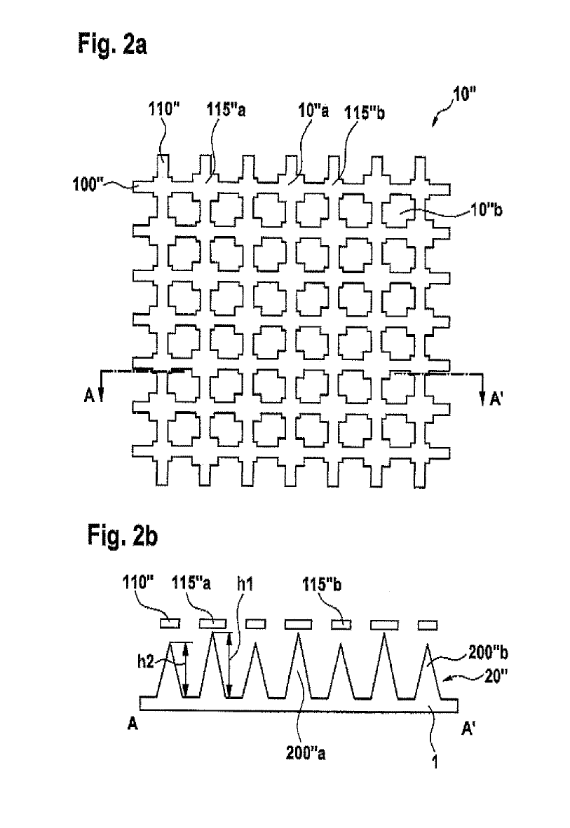

FIGS. 2a,b are schematic representations for the explanation of a second embodiment of the production process according to the disclosure for a microneedle arrangement, to be precise FIG. 2a is a plan view of an etching grid and FIG. 2b is a cross-sectional view of the etching grid and of the microneedle arrangement resulting from it along the line A-A′ from FIG. 2a.

In the case of the second embodiment according to FIG. 2, reference sign 10″ denotes an etching mask of oxide, which likewise has horizontal grid bars 100″ and vertical grid bars 110″, which are arranged in an orthogonal form. In the case of the etching mask 10″, the grid crossing regions are denoted by 10″a and the grid openings are denoted by 10″b.

As a difference from the first embodiment described above, in the case of the second embodiment the square reinforcing regions 115″a and 115″b at the grid crossing regions 10″a vary with regard to their surface area. For instance, in the case of the present example, the first reinforcing regions 115″a have a larger surface area than the second reinforcing regions 115″b.

If the anisotropic/isotropic etching process described above is applied in the case of such an etching mask 10″, higher, thicker microneedles 200″a and narrower, lower microneedles 200″b are created, as represented in FIG. 2b. The higher, thicker microneedles 200″a form under the larger reinforcing regions 115″a, and the narrower, lower microneedles 200″b form under the smaller reinforcing regions 115″b.

After the anisotropic etching process, the narrower and thicker microneedles still have in fact the same height, but during the isotropic etching process the narrower microneedles are etched more quickly and lose height in comparison with the thicker microneedles, so that the microneedle arrangement 20″ shown in FIG. 2b is obtained.

A typical size for the thicker, higher microneedles 200″a is a height h1=180 μm, a typical order of size for the narrower, lower microneedles 200″b is a height h2=120 μm. Tests have shown that extremely efficient piercing characteristics can be achieved if the difference in height between the microneedles 200″a and 200″b is in the range of 20-50%.

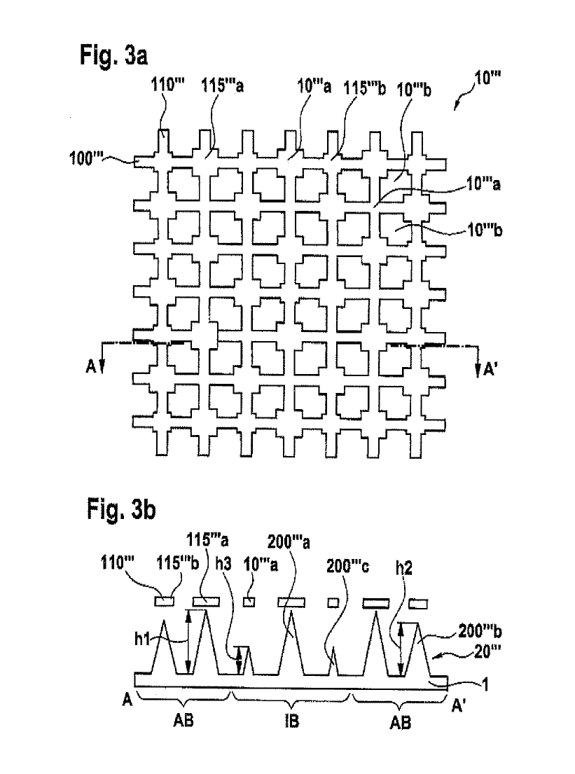

FIGS. 3a,b are schematic representations for the explanation of a third embodiment of the production process according to the disclosure for a microneedle arrangement, to be precise FIG. 3a is a plan view of an etching grid and FIG. 3b is a cross-sectional view of the etching grid and of the microneedle arrangement resulting from it along the line A-A′ from FIG. 3a.

In the case of the third embodiment, the etching mask 10′″ likewise has horizontal grid bars 100′″ and vertical grid bars 110′, which are arranged in the orthogonal grid form already described.

In the case of the etching mask 10′″, at the grid crossing regions 10′″a first reinforcing regions 115′″a with a larger area or second reinforcing regions 115′″b with a smaller area are provided and at certain grid crossing regions 10′″a no reinforcing regions at all are provided. The latter grid crossing regions lie in the inner region IB of the etching mask 10′″ or of the resulting microneedle arrangement 20′″ with the grid openings 10′b.

As represented in FIG. 3b, three different types of microneedle 200′″a, 200′b and 200′c can be produced in the microneedle arrangement 20′″ by means of the etching mask 10′″ in the etching process already described above. The first microneedles 200′″a are thicker needles with a greater height h1 of typically 180 μm, the second microneedles 200′″b are narrower, lower microneedles with a height h2 of typically 120 μm, and the third microneedles 200′″c are very narrow, very low microneedles with a height h3 of typically 90 μm.

As shown in FIGS. 3a,b, the three microneedles 200′″c are not arranged in the outer region AB of the microneedle arrangement 200′″, but in the inner region IB thereof. In other words, they are shielded from the outer region AB by the first microneedles 200′″a, so that, for example in the case of porous microneedles of silicon, the risk of breakage due to canting can be reduced or avoided.

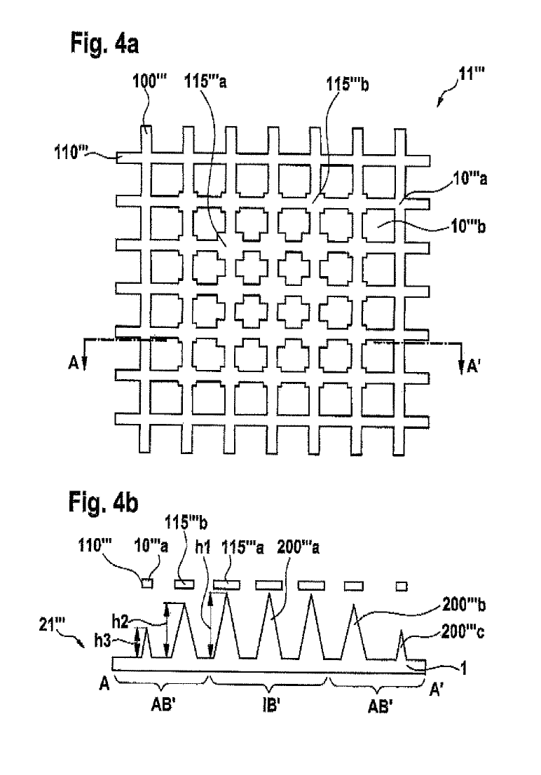

FIGS. 4a,b are schematic representations for the explanation of a fourth embodiment of the production process according to the disclosure for a microneedle arrangement, to be precise FIG. 4a is a plan view of an etching grid and FIG. 4b is a cross-sectional view of the etching grid and of the microneedle arrangement resulting from it along the line A-A′ from FIG. 4a.

In the case of the fourth embodiment, the etching mask 11′″ likewise has horizontal grid bars 100′″ and vertical grid bars 110′, which are arranged in the orthogonal grid form already described.

In the case of the etching mask 11′″, at the grid crossing regions 10′a first reinforcing regions 115′″a with a larger area or second reinforcing regions 115′b with a smaller area are provided and at certain grid crossing regions 10′″a no reinforcing regions at all are provided. The latter grid crossing regions lie in the outer region AB′ of the etching mask 11′ or of the resulting microneedle arrangement 21′″ with the grid openings 10′b.

As represented in FIG. 4b, three different types of microneedle 200′″a, 200′b and 200′c can be produced in the microneedle arrangement 21′″ by means of the etching mask 11′″ in the etching process already described above. The first microneedles 200′″a are thicker needles with a greater height h1 of typically 180 μm, the second microneedles 200′″b are narrower, lower microneedles with a height h2 of typically 120 μm, and the third microneedles 200′″c are very narrow, very low microneedles with a height h3 of typically 90 μm.

As shown in FIGS. 4a,b, the height of the microneedles 200′a, 200′″b, 200′″c increases in stages from the outer region AB′ to the inner region IB′.

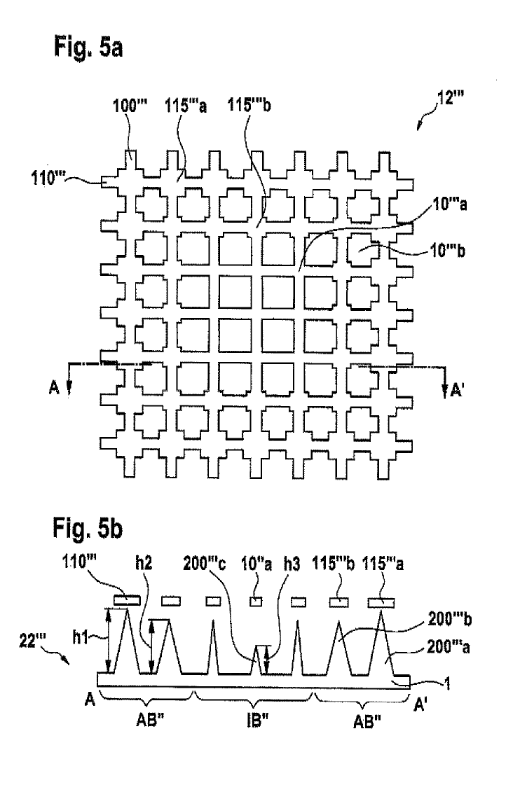

FIGS. 5a,b are schematic representations for the explanation of a fifth embodiment of the production process according to the disclosure for a microneedle arrangement, to be precise FIG. 5a is a plan view of an etching grid and FIG. 5b is a cross-sectional view of the etching grid and of the microneedle arrangement resulting from it along the line A-A′ from FIG. 5a.

In the case of the fifth embodiment, the etching mask 12′ likewise has horizontal grid bars 100′″ and vertical grid bars 110′, which are arranged in the orthogonal grid form already described.

In the case of the etching mask 12′″, at the grid crossing regions 10′a first reinforcing regions 115′″a with a larger area or second reinforcing regions 115′b with a smaller area are provided and at certain grid crossing regions 10′″a no reinforcing regions at all are provided. The latter grid crossing regions lie in the inner region IB″ of the etching mask 12′ or of the resulting microneedle arrangement 22″ with the grid openings 10′b.

As represented in FIG. 5b, three different types of microneedle 200′″a, 200′b and 200′c can be produced in the microneedle arrangement 20′″ by means of the etching mask 12′″ in the etching process already described above. The first microneedles 200′″a are thicker needles with a greater height h1 of typically 180 nm, the second microneedles 200′″b are narrower, lower microneedles with a height h2 of typically 120 nm, and the third microneedles 200′″c are very narrow, very low microneedles with a height h3 of typically 90 nm.

As shown in FIGS. 5a,b, the height of the microneedles 200′a, 200′″b, 200′″c decreases in stages from the outer region AB″ to the inner region IB″.

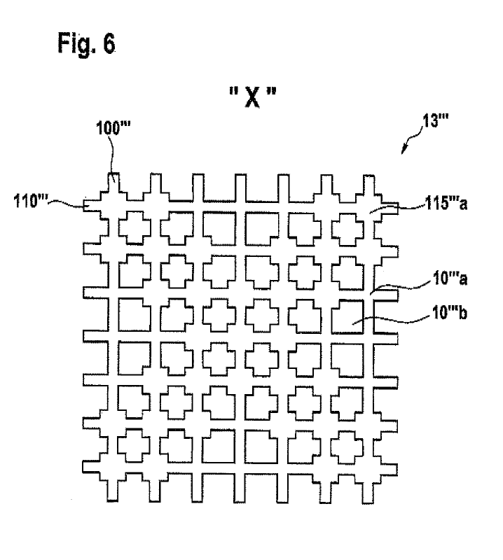

FIG. 6 is a plan view of an etching grid for the explanation of a sixth embodiment of the production process according to the disclosure for a microneedle arrangement.

In the case of the sixth embodiment, the etching mask 13′″ likewise has horizontal grid bars 100′″ and vertical grid bars 110′, which are arranged in the orthogonal grid form already described.

In the case of the etching mask 13′″, at the grid crossing regions 10′a first reinforcing regions 115′″a are provided and at certain grid crossing regions 10′a no reinforcing regions at all are provided. The first reinforcing regions 115′″a are arranged in such a way that the etching mask assumes an “X” pattern. This “X” pattern is transferred during the etching to the corresponding microneedle arrangement, which then can be used for example in conjunction with a tattooing fluid for the tattooing of a human or animal body.

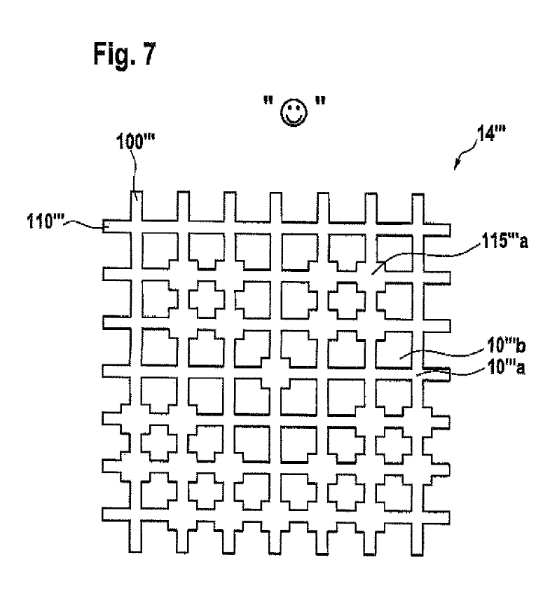

FIG. 7 is a plan view of an etching grid for the explanation of a seventh embodiment of the production process according to the disclosure for a microneedle arrangement.

In the case of the seventh embodiment, the etching mask 14′″ likewise has horizontal grid bars 100′ and vertical grid bars 110′″, which are arranged in the orthogonal grid form already described.

In the case of the etching mask 13′″, at the grid crossing regions 10′a first reinforcing regions 115′″a are provided and at certain grid crossing regions 10′a no reinforcing regions at all are provided. The first reinforcing regions 115′″a are arranged in such a way that the etching mask assumes a “ [symbol] ” pattern. This “ [symbol] ” pattern is transferred during the etching to the corresponding microneedle arrangement, which then can likewise be used for example for tattooing.

Although the present disclosure has been described above on the basis of preferred exemplary embodiments, it is not restricted to these but can be modified in various ways.

Although in the case of the embodiments described above certain materials have been described, for example silicon as the substrate and oxide for the etching mask, the present disclosure is not restricted to these but can be applied to any materials that have corresponding etching characteristics or a corresponding etching selectivity.

The grid form of the etching mask is also not restricted to the orthogonal, square form shown but can in principle be applied to any forms of grid. The reinforcing regions at the grid crossing regions do not have to be square but may assume any geometry, for example also a round geometry or a rhomboidal geometry, etc.

Furthermore, the present disclosure is not restricted to porous microneedles of silicon but can in principle be applied to any microneedles that can be produced in an etching process using an etching mask.

1. A production process for a microneedle arrangement, comprising:
forming an etching mask in grid form, with grid bars with corresponding grid crossing regions and grid openings in between on a substrate;
carrying out an etching process to form the microneedle arrangement on the substrate using the etching mask; and
removing the etching mask,
wherein the etching mask in grid form has at least some of the grid crossing regions flat reinforcing regions, which extend beyond the grid bars.

2. The production process according to claim 1, wherein the etching mask in grid form has at least first and second flat reinforcing regions of different area extents and the etching process being carried out in such a way that the microneedle arrangement has corresponding first and second microneedles of different first and second heights, respectively.

3. The production process according to claim 1, wherein the substrate is a silicon substrate and the etching mask is formed as an oxide mask.

4. The production process according to claim 1, wherein some of the grid crossing regions possess no flat reinforcing regions.

5. The production process according to claim 4, wherein the grid crossing regions that possess no flat reinforcing regions lie in an inner region of the etching mask in grid form.

6. A microneedle arrangement comprising:
a substrate; and
a plurality of microneedles formed on the substrate, said plurality of microneedles possessing different heights with respect to each other.

7. The microneedle arrangement according to claim 6, wherein:
said plurality of microneedles including at least first and second microneedles, and
said first and second microneedles respectively possess first and second heights that differ from each other.

8. The microneedle arrangement according to claim 7, wherein a difference in height of the first and second heights lie in the range of 20% to 50%.

9. The microneedle arrangement according to claim 6, wherein:
said plurality of microneedles includes at least first, second, and third microneedles, and
said first, second, and third microneedles respectively possess first, second, and third heights that differ from each other.

10. The microneedle arrangement according to claim 9, wherein the third microneedles has a third height, which is the lowest height, and the third microneedles being provided only in an inner region of the microneedle arrangement.

11. The microneedle arrangement according to claim 6, wherein the microneedle arrangement is configured for tattooing of a human body or an animal body.

12. A microneedle arrangement including a plurality of microneedles which are formed on a substrate and have different heights with respect to each other, said microneedle arrangement being configured for tattooing a human body or an animal body.
